(19) United States
(12) Patent Application Publication
(10) Pub. No.: US 2005/0220714 A1
(43) Pub. Date: Oct. 6, 2005

(54) Agents for use in magnetic resonance and optical imaging

(76) Inventors: Susan Kauzlarich, Davis, CA (US); Angelique Louie, Woodland, CA (US)

Correspondence Address: MORRISON & FOERSTER LLP, 425 MARKET STREET, SAN FRANCISCO, CA, 94105-2482, US

(21) Appl. No.: 11/086,530

(22) Filed: Mar. 21, 2005

Related U.S. Application Data
(60) Provisional application No. 60/559,374, filed on Apr. 1, 2004.

(51) Int. Cl. A61K 49/00; H01L 21/00

(52) U.S. Cl. 424/9.32; 424/9.6; 438/1

(57) ABSTRACT

Semiconductor nanoparticles are doped with paramagnetic ions to serve as dual-mode optical and magnetic resonance imaging (MRI) contrast agents. These nanoparticles can be constructed in smaller diameters than typical MRI agents. The dual-modality nature allows the particles to be used for in vivo imaging by MRI, and then followed by histology with optical imaging techniques.

### CROSS-REFERENCE TO RELATED APPLICATIONS

This application claims priority to U.S. provisional application No. 60/559,374 filed on Apr. 1, 2004 which is hereby incorporated by reference in its entirety.

### FIELD OF THE INVENTION

The present invention relates generally to agents for use in imaging of biological samples. In particular, it relates to imaging agents with properties which allow their use in magnetic resonance imaging (MRI) as well as optical imaging techniques, both in vivo and in vitro.

### BACKGROUND OF THE INVENTION

Luminescent nanoparticles have the potential to overcome problems encountered by organic small molecules in certain fluorescent tagging applications by combining the advantages of high photobleaching threshold, good chemical stability, and readily tunable spectral properties.

Nanoparticles are small particles with a typical size of a few nanometers (Wolfgang et al., 2003). Due to their discrete energy level similar to that of atoms, nanoparticles are often thought of as artificial atoms (Warburton, et al., 1997). The spacing between energy levels in a nanoparticle can be tuned by controlling its size. For example, Cadmium Selenide (CdSe) nanoparticles show all fluorescence colors in the visible region by controlled adjustment of their size. Due to many advantages over traditional dyes, nanoparticles are ideal fluorophores for molecular biotechnology and bioengineering applications (Bruchez et al., 1998, Chan et al., 1998). The application of luminescent nanoparticles in vivo is limited, however, due to the shallow depth of penetration of light through tissues. Clinical imaging methodologies such as magnetic resonance imaging (MRI) offer unlimited depth of interrogation, but poorer resolution than optical methods. Imaging probes that could be detected by both MRI and optical methods could combine the strengths of both imaging technologies to provide information about the subject of interest.

Nanoparticles of various compositions have been described that are “doped” with metals, such as manganese and copper in order to alter their luminescent properties, or to develop magnetic storage devices (Bol et al., 2002, Borse et al., 1999, Bargik-Chory et al., 2003, Mikulec et al., 2000, Levy et al., 1996, Ethiraj et al., 2003, Levy et al., 1999, Dinsmore et al., 2000). “Doped” refers to the incorporation of the metal into the nanoparticle. However, these nanoparticles are limited by the low level of incorporation of the magnetic material, and a possible decrease in the photoluminescent efficiency of the nanoparticle.

There is thus a need to provide dual mode imaging agents which can be visualized by both magnetic resonance imaging and optical imaging.

### SUMMARY OF THE INVENTION

The present invention provides dual mode imaging agents which can be visualized by both magnetic resonance imaging (MRI) and optical imaging. In one format, the imaging agent is a quantum dot.

In one embodiment, the imaging agent includes an optically active luminescent nanoparticle core and a shell comprising a magnetic material. The luminescent nanoparticle can be, e.g., a Group II-VI nanoparticle, a Group III-V nanoparticle or a Silicon nanoparticle. As used herein, a Group II-VI nanoparticle refers to a nanoparticle which contains an element from Group II and an element from Group VI, e.g., Zinc Sulfide (ZnS) contains Zinc from Group II and Sulfur from Group VI, so ZnS is a Group II-VI nanoparticle. As used herein, a Group III-V nanoparticle refers to a nanoparticle which contains an element from Group III and an element from Group V. This applies to other combinations as well, e.g. a Group II-IV nanoparticle refers to a nanoparticle which contains an element from Group II and an element from Group IV, etc.

The Group II element can be Zinc, Cadmium, or Manganese and the Group VI element can be Sulfur, Selenium, or Tellurium. The magnetic material can be Manganese (II), Iron (III), Gadolinium, or other magnetic lanthanides. The shell can also contain a matrix, which for example can be made of Zinc Sulfide, Cadmium Sulfide and Silicon. In one embodiment the core is Cadmium Selenide, the matrix is Zinc Sulfide, and the magnetic material is Manganese (II). In another embodiment of the invention, the core is a Silicon nanoparticle core and the shell is a SiOₓ shell, produced by silanization of the surface.

The imaging agent of the invention can also have a water soluble coating. Such water soluble coatings can be formed by treatment of the imaging agent with a treatment or agent such as: mercaptoalkanoic acid ligands, PEG, silanization, phospholipid micelles, organic dendrons, cysteines, or amphipathic polymers. Another aspect of the invention can be the addition of a moiety attached to the shell including a functional group such as amine, carboxy, and thiol.

A further aspect of the present invention is a method for imaging a biological sample with the imaging agents of the invention. This method can include contacting the biological sample with the imaging agent of the invention as described, and detecting the imaging agent by optical detection and MRI detection. Alternatively, only optical detection or MRI detection can be performed.

The invention further provides a luminescent nanoparticle “core” surrounded by a “shell” into which a magnetic material can be incorporated. The advantages of using these agents are 1) the magnetic material can be incorporated at a much higher concentration than can be incorporated into the core, 2) the shell improves the photoluminescent efficiency of the core, and 3) the magnetic material does not interfere with the crystal lattice structure of the core.

The nanoparticles are dual-mode agents, being visible via both optical methods and magnetic resonance imaging (MRI). This advance permits the combination of positive features associated with optical imaging and magnetic resonance imaging. The invention embraces a variety of nanoparticle compositions including, e.g., Silicon-based nanoparticles and cadmium-based nanoparticles.

The nanoparticle compositions further provide optical read-out based on their ability to produce bright luminescence of a form that does not fade over time or with repeated irradiations. This non-bleaching luminescence makes the compositions useful for imaging applications requiring repeated interrogation of regions of interest. The optical imaging permits collection and assessment of tissue in histological investigation.

The nanoparticle compositions also provide magnetic resonance imaging based on the presence of metals which alter the signal and provide contrast enhancement. The nanoparticle compositions can be constructed in much smaller size diameters than is typical for MRI agents. The compositions can thus be used to obtain in vivo magnetic resonance images as a complement to the optical imaging at the histological stage. For instance, the compositions can be used to confirm, via optical imaging, that biopsied tissue comes from the same area of interest as identified by magnetic resonance imaging.

The nanoparticle compositions of the invention are water soluble, and may be coated with non-toxic groups. The compositions may also be attached to specific molecules of interest to allow targeting to specific cell or tissue types, or to allow binding of other molecules.

The advantages of these nanoparticles are several fold. As optical imaging agents, their non-bleaching luminescence is well described and renders them extremely useful for imaging applications requiring repeated interrogation of regions of interest. Attachment to specific molecules of interest can allow targeting of the particles to specific cell or tissue types, or to allow binding to other molecules. With the magnetic component, these particles can be used to obtain in vivo images using MRI, and then tissue can be collected for histological investigation using optical imaging. This method would be invaluable, for example, to allow a clinician to be certain that biopsied tissue came from the area of interest identified in an MRI image.

### BRIEF DESCRIPTION OF THE DRAWINGS

FIG. 1 depicts the synthesis and termination procedure for Silicon nanoparticles.

FIG. 2 depicts a schematic of Core/Shell luminescent and magnetic nanoparticles.

FIG. 3 depicts a method of incorporating a metal onto the surface of a Silicon nanoparticle.

FIG. 4 depicts an alternate method of incorporating a metal onto a nanoparticle where the metal is trapped in the oxide layer at the surface of the nanoparticle.

FIG. 5A depicts the surface modification of nanoparticles with amphipathic copolymer. B depicts a triblock copolymer composed of methacrylic acid (23%), butylacylate, and ethylacylate (77%).

FIG. 6 depicts the IR spectra of Mn₂(μ-SeMe)₂—(CO)₈ and Mn(CO)₅Br.

FIG. 7 depicts in A the morphology of the synthesized Cd₁₋ₓMnₓSe, B depicts the energy disperse spectrum. C depicts the surface of a nanoparticle covered with an organic layer of TOPO molecules.

FIG. 8 depicts the room-temperature absorption and emission spectra of Mn-doped CdSe nanoparticles.

FIG. 9 depicts the low-temperature EPR of Mn-doped CdSe nanoparticles before and after surface-exchange with pyridine.

FIG. 10 depicts in A the absorption spectra of CdSe and core/shell. B depicts the PL spectra of CdSe and core/shell CdSe/Zn_{0.99}Mn_{0.01}S nanoparticles (excited at 400 nm). C depicts the TEM showing the CdSe/Zn_{0.99}Mn_{0.01}S nanoparticles and the average particle size is about 4.3 nm. (The image is not clear, so the particle size measured may be not accurate.) D depicts the low-temperature (4 K) EPR spectrum of CdSe/Zn_{0.99}Mn_{0.01}S.

FIG. 11 depicts in A the absorption spectra of CdSe and core/shell CdSe/Zn_{0.97}Mn_{0.03}S nanoparticles. B depicts the PL spectra of CdSe and core/shell CdSe/Zn_{0.97}Mn_{0.03}S nanoparticles (excited at 400 nm). C depicts the TEM image showing monodispersed and uniform structure. Average particle size is 6.3 nm. D depicts the low-temperature (4 K) EPR spectrum of CdSe/Zn_{0.97}Mn_{0.03}S.

FIG. 12 depicts in A CdSe/Cd₁₋ₓMnₓS nanoparticles prepared from a nominal Mn-doping level of 1% (molar ratio of Manganese to total Cd, the same as follows) and 6 mL H₂S gas for 1 mmol Cd. (a) absorption spectra, (b) photoluminescent spectra. B depicts CdSe/Cd₁₋ₓMnₓS nanoparticles prepared from a nominal Mn-doping level of 2% and 7 mL H₂S gas for 1 mmol Cd. (a) absorption spectra, (b) photoluminescent spectra (excited at 400 nm). C depicts CdSe/Cd₁₋ₓMnₓS nanoparticles prepared from a nominal Mn-doping level of 3% and 8 mL H₂S gas for 1 mmol Cd. (a) absorption spectra, (b) photoluminescent spectra.

FIG. 13 depicts TEM results for the sample with a nominal composition of molar ratio of Si:Zn:=41:1:0.08. (a) Micrograph, (b) EDS spectra.

FIG. 14 depicts in A the absorption and emission spectra, B depicts the low-temperature EPR spectrum, C depicts the NMR results, showing T1 relaxation.

FIG. 15 depicts in A the comparison of the emission profiles between the dye of rhodamine 6 G and core/shell CdSe/ZnS nanoparticles. B depicts the XRD pattern which shows the wurtzite structure of CdSe nanoparticles.

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art.

“Nanoparticle” refers to a microscopic particle whose size is measured in nanometers, with a typical size of a few nanometers to 100 nm.

“Luminescent” refers to a material that emits light of a given wavelength after irradiation with light of another wavelength.

“Optically active” refers to a material that is capable of luminescing.

“Core” refers to the center part of the nanoparticle and is typically single phase.

“Shell” refers to any additional layers of material with a different composition than the core. These additional layers need not be contiguous to completely surround the core.

“Magnetic” and “Paramagnetic” refer to magnetic properties of materials that allow them to be visible by MRI.

“Matrix” refers to the basic elements comprising the crystalline lattice of the shell, minus the magnetic dopant material.

“Dopant” refers to a chemical element added in small amounts to a material to modify properties of the material.

“Quantum dot” refers to semiconductor nanoparticles, which are known in the art as optical imaging agents (Gao et al., 2002, Chan, et al., 2002, Chan et al., 1998, Kulkarni et al., 2001, and Sutherland et al., 2002). They are brightly luminescent in a manner that does not fade over time and repeated irradiation. The particles described in the literature are largely CdS type particles, in order to apply them to biological systems they must be coated to render them non-toxic (Larson et al., 2003, Dabbousi et al, 1997, Hines et al., 1996, Peng et al., 1997, Pathak et al., 2001, and Jaiswal et al., 2003).

The purpose of the present invention is the use of semiconductor nanoparticles that are doped with metal to alter the signal in magnetic resonance imaging (MRI). These doped quantum dots will give contrast enhancement in MRI, and can be coated with non-toxic groups to allow use in biological systems.

Nanoparticles of the invention of “core-shell” design integrate a luminescent core with a surface shell incorporating a magnetic transition metal ion such as Manganese(II), Lanthanides(III) or Iron(II or III). The nanoparticles are detectable by both optical methods and magnetic resonance imaging. Overall size may vary from 1-50 nm. Core size typically is less than 20 nm, shell size may vary from one to several monolayers thick (below 1 nm to no larger than 50 nm).

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

Core materials are typically luminescent nanoparticles such as (but not limited to) Group II-VI nanoparticles, Group III-V nanoparticles, or Silicon. Emission wavelength for these materials is size dependent. Semiconductor nanoparticles have been exploited as biological indicators because of their narrow, tunable, symmetric emission spectrum. The benefit of being able to excite with broad excitation energy and obtain multicolored fluorescent output has been one of the advantages of semiconductor nanoparticles. In addition, emission is environmentally stable as the production of photons stems from a band-gap process rather the singlet-singlet transition typical for small molecule fluorophores. The nanocrystalline semiconductors that are probably the best characterized for biological applications to date are the Group II-VI nanoparticles (II=Zinc, Cadmium, Manganese; VI=Sulfur, Selenium, Tellurium). The synthesis of these nanoparticles is relatively straightforward and allows size control. In addition, methods for surface capping are well-documented. It has been demonstrated that the brightest, most stable nanoparticles with the best synthetic control over size are CdSe/ZnS core/shell nanoparticles. The ZnS passivates the surface, removing non-radiative defects and also diminishes photodecomposition of the nanoparticle.

There is currently significant effort towards investigating the biological fate and the possible toxicity of these nanoparticles, because of the presence of cadmium. (Kirchner et al., 2005) Nanoparticles containing cadmium are not toxic but the concern is that breakdown of the nanoparticles in vivo would release free cadmium which is highly toxic. Nanoparticles of Silicon would avoid this possible toxicity in that even free Silicon is not toxic in the body. Studies on porous Silicon films have shown that the primary decomposition product is orthosilicic acid (Si(OH)₄) which is a component for normal bone and connective tissue homeostasis. The additional Si(OH)₄ does not adversely affect homeostasis (Anderson et al., 2003). Silicon nanoparticles prepared from porous Silicon and gas phase decomposition of silane have recently been conjugated with a 5′-amino-modified oligonucleotide (Wang et al., 2004), embedded in phospholipid bilayers (Jang et al., 2003), and grafted with poly(acrylic acid) (Li et al, 2004) in order to utilize these nanoparticles as biological probes. These studies suggest that Silicon nanoparticles are highly promising for biological applications, particularly for applications where Cadmium-containing nanoparticles may not be applicable.

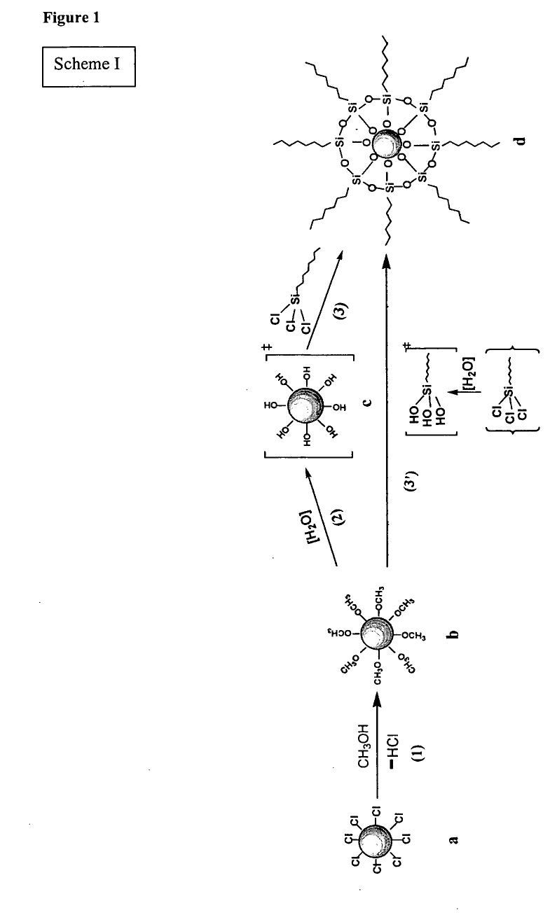

Presented herein is a high yield, facile, solution synthesis of Silicon nanoparticles as well as a silanization termination forming siloxane surface-coated Silicon nanoparticles (Zou et al., 2004, Liu et al., 2002). Silanization has been widely used to alter the surface characteristics of various kinds of nanoparticles, such as CdSe nanoparticles and Gold nanoparticles, with good success (Gerion et al., 2001, Parak et al., 2003). Silanization has also been used to alter the surface of nanostructured Silicon and Silicon nanoparticles (Padadimitrakopoulos et al., 1999, Ji et al., 2001, Li et al., 2004). The synthesis and termination procedure is outlined in Scheme I shown in FIG. 1. The synthesis leading to the primary Cl-terminated Silicon nanoparticles (a) has been published (Baldwin et al., 2002) and involves facile reduction of SiX₄ (X=halogen) to produce halide terminated Silicon nanoparticles (a) that can be further terminated by various organic moieties. Starting with these halide terminated Silicon nanoparticles, a silanization method was applied in situ to obtain an oxide coating that contains an organic moiety capable of further functionalization to produce a core-shell nanoparticle, Si/SiO₂—R. Silicon nanoparticles were first treated with methanol to substitute the halide (Cl) with methoxy groups at the surface (b). From there, two routes are possible to silanize the nanoparticles as shown below (2-3 or 3′) both of which involve reaction with octatrichlorosilane and water, but in different order of reaction. The (2-3) route generates hydroxyl-terminated Silicon nanoparticles (c) as an intermediate whereas (3′) generates octatrihydoxysilane. Either pathway (2-3 or 3′) is possible and cannot be distinguished experimentally. Both pathways result in a long-chain hydrocarbon surface coating (d) that significantly enhances the stability of nanoparticles with no effect on optical properties of the Silicon core.

Typical shell materials are ZnS and CdS doped with paramagnetic materials discussed above. These shells, in principle, can be put on any of the cores provided above (Group II-VI nanoparticles, Group III-V nanoparticles, or Silicon). In addition, silanization of the core also affords a surface that will allow for doping the shell (now SiOₓ) with paramagnetic materials.

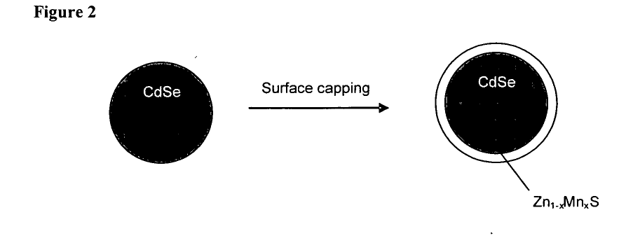

The present invention involves synthesis of CdSe/Zn₁₋ₓMnₓS nanoparticles with a range of levels of Manganese doping from 1-10%. The general structure of these nanoparticles is illustrated in the schematic shown in FIG. 2. (Doping Mn²⁺ into ZnS has great influence on the optical properties of ZnS nanoparticles. Besides the appearance of a new peak caused by the ⁴T₁₋₆A₁ emission of Mn²⁺ (Cao et al., 2002), the luminescence quantum efficiency of Zn₁₋ₓMnₓS also depends on the concentration of Mn²⁺.) The luminescence quantum efficiency increases before the doping level of the Mn²⁺ content reaches a data ranging differently from 0.12-4 at. % based on various reports (Khosravi et al., 1995, Sooklal et al., 1996, Leeb et al., 1999). Doping of ZnS nanoparticles has been described in the literature at levels up to 3%. Higher doping tends to eliminate luminescence properties (Khosravi et al., 1995, Sooklal et al., 1996, Leeb et al., 1999). Because in the present invention the magnetic material is incorporated into the shell, there is not as much concern with loss of luminescence for this material. Since the magnetic material is not being directly incorporated into the core, it is hypothesized that Manganese levels in the shell will not affect luminescence from CdSe.

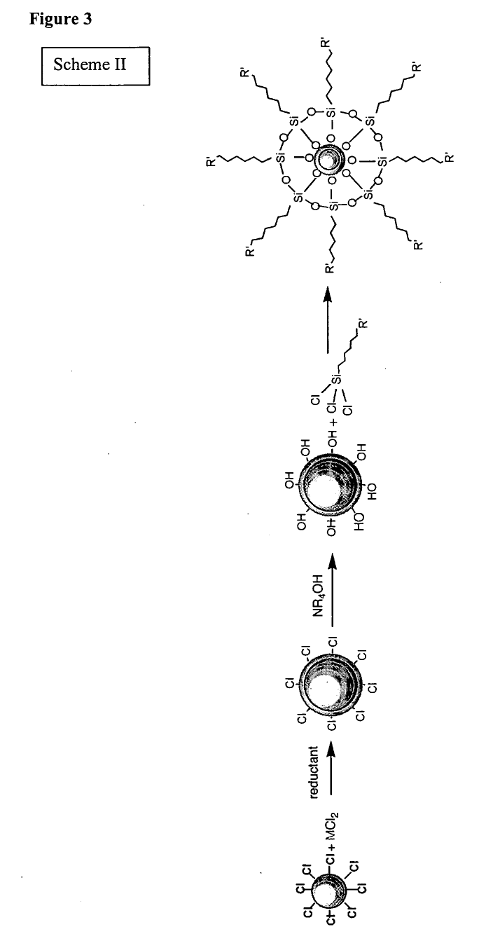

There are two possible places in the synthesis in which to incorporate a metal on to the Silicon nanoparticles surface, each places the metal dopant at different positions relative to the surface. The first method involves obtaining the chloride terminated Silicon nanoparticles, and then metal halides such as (but not limited to) MnCl₂ (or FeCl₂) can be added to the solution so as to react further with the surface to produce Si₁₋ₓMₓ—Cl nanoparticles (M=Mn, Fe) with the M trapped at the Silicon nanoparticle surface (Scheme II shown in FIG. 3).

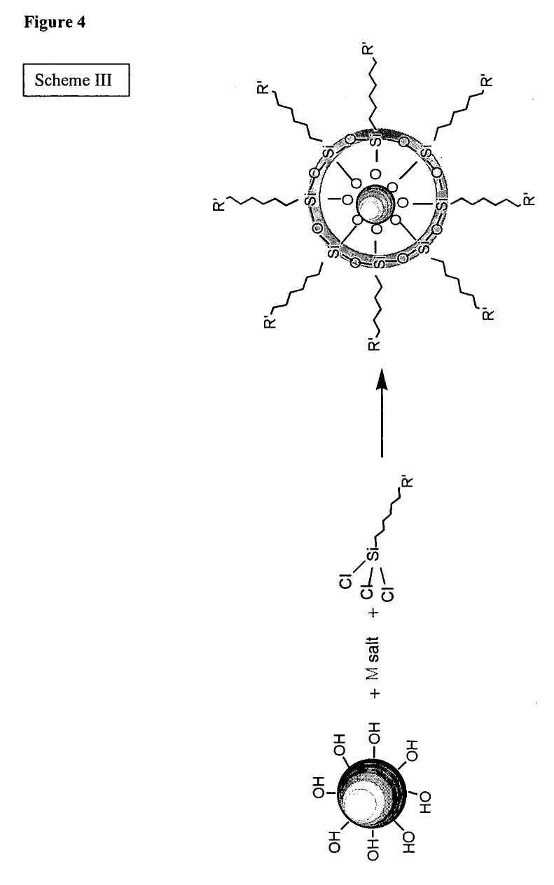

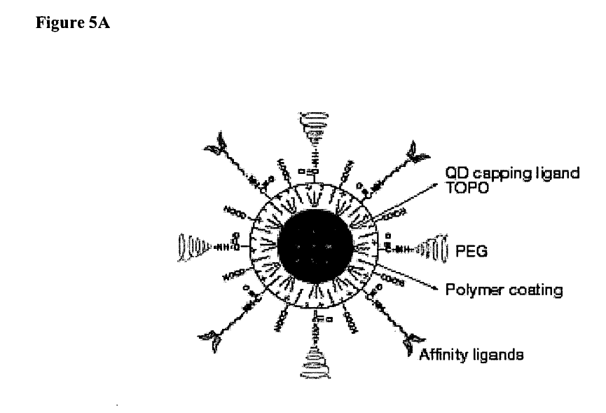

As an alternate method, the metal, M, may be doped into the oxide surface as Si/Si₁₋ₓMₓO₂—R by reacting the metal with the Silicon hydroxide surface of the nanoparticle and then silanating the surface. This may be possible with various M containing salts, such as Manganese acetate, or organometallics, in conjunction with the silanation agent. This introduces the magnetic dopants prior to surface termination to trap them in the oxide layer at the surface of the nanoparticles (Scheme III shown in FIG. 4).

The chain length of the hydrocarbon can be altered if it is determined to interfere with magnetic properties of the nanoparticles. Depending on the concentration of Manganese which can be incorporated, the nanoparticles may affect T₁ or T₂ more strongly. This can be empirically determined as the nanoparticles are tested for their magnetic properties. In studies of core-shell CdS/ZnMnS particles, sufficient amounts of Manganese have been incorporated into shells to generate nanoparticles visible by MRI with relaxivities typical for Manganese agents.

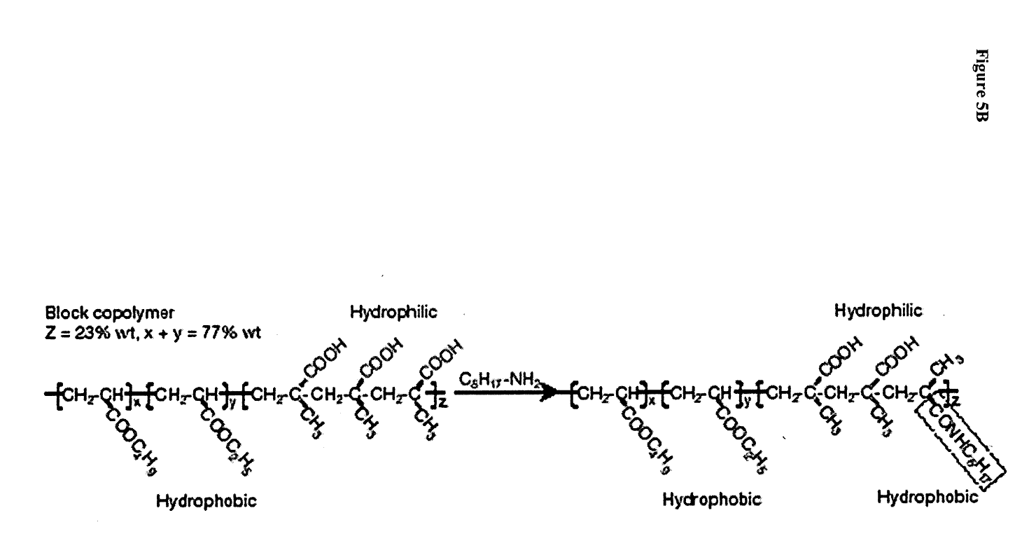

There are a number of literature reported methods to add a water soluble coating to the nanoparticles by using mercaptoalkanoic acid ligands (Warren et al., 1998), silanization (Gerion et al., 2001), phospholipid micelles (Dubertret et al., 2002), organic dendrons Wang et al., 2002), cysteines Sukhanova et al., 2002), or amphiphilic polymers (Gao et al., 2004). FIG. 5A depicts the surface modification of nanoparticles with amphipathic copolymer. Self-organized surfactant molecules bind its hydrophobic moiety to the surface of nanoparticles leaving its hydrophilic moiety towards outside. A triblock copolymer, shown in FIG. 5B, of polybutylacylate-co-polyethylacylate-co-polymethacrylic acid with an average molecular weight of ˜100,000 daltons (below left) is derivatized with octylamine. The polymer is dissolved with dimethylformamide (DMF) and reacted with n-octylamine at a polymer/octylamine molar ratio of 1:40, using ethyl-3-dimethylamino propyl carbodiimide (EDAC, threefold excess of n-octylamine) as a cross-linking reagent. The reaction mixture is dried and the resulting oily liquid is precipitated with water and is rinsed with water fives times to remove excess EDAC and other by-products. After drying, the octylamine-grafted polymer is resuspended in an ethanol-chloroform mixture. About 25% of carboxylic acids groups are modified. Polymer is then reacted with nanoparticles in a ratio of 5-10:1 (polymer:nanoparticle). The mixture is dried and the encapsulated dots dissolved in ethanol and purified by gel filtration using PBS buffer solution (PH=8˜9). The encapsulated nanoparticles can then be conjugated to biomolecules (e.g. affinity targets) or with additional polymers to increase solubility, (such as PEG).

Surfaces of the nanoparticles may be terminated with a functional group such as amine, carboxyl or thiol. These can then be conjugated to biomolecules using traditional crosslinking methods.

The present invention may be used in conjunction with the synthesis of CdSe/Zn₁₋ₓMnₓS at 1-2% Manganese doping levels. Particle uniformity and size can be verified by TEM. Addition of the ZnMnS shell increases photoluminescence of the particles. Presence of Manganese can be verified by EPR. The ability of Manganese doping to affect magnetic resonance of nanoparticles can be confirmed in silica coated ZnMnS particles, where 50 nm particles of <1% Manganese are found to have a relaxivity of 8.357 mM⁻¹/s⁻¹. This indicates that they will be visible by MRI.

One example of a possible use for the present invention is to conjugate a lymphoma specific petidomimetic (ligand 2A) to the imaging agent of the invention in order to label lymphomas. An ideal usage for the imaging agent of the invention would be to label tumors, use MRI to identify the location of the tumors, and then during surgical resection, use optical methods to be sure that all positively labeled cells are removed. Another application would be to specifically target the imaging agent of the invention to atherosclerotic plaques, use MRI to identify possible lesions and then use optical detection for image-guided intervention.

### DESCRIPTION OF PREFERRED EMBODIMENTS

### EXAMPLE 1

### Cd₁₋ₓMnₓSe

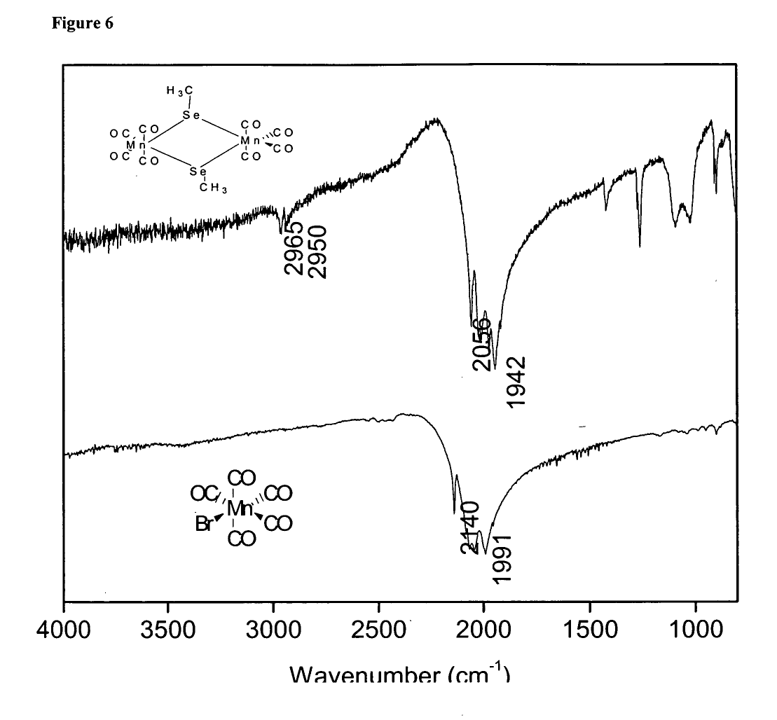

Cd₁₋ₓMnₓSe was synthesized from the organometallic complex Mn₂(μ-SeMe)₂—(CO)₈ via a high-temperature pyrolysis process (Mikulec et al., 2000). This method was followed and Mn-doped CdSe nanoparticles were synthesized. The compound Mn₂(μ-SeMe)₂—(CO)₈ was synthesized, which was confirmed by some of the characterization results such as infrared spectrum and chemical analysis. FIG. 6 shows the IR spectra of both the compound Mn₂(μ-SeMe)₂—(CO)₈ and starting material of Mn(CO)₅Br.

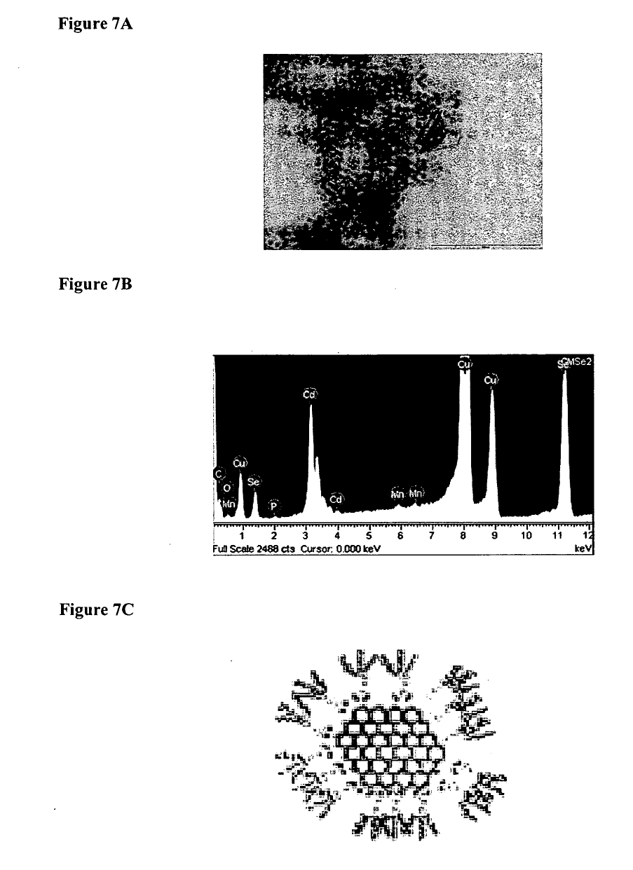

Cd₁₋ₓMnₓSe nanoparticles were then prepared in a mixture of tri-n-octylphosphine oxide (TOPO) and hexadecylamine (HDA) by use of the synthesized compound of Mn₂(μ-SeMe)₂—(CO)₈ as Manganese source. FIG. 7 shows the results from TEM measurement. As shown in FIG. 7A, the synthesized Cd₁₋ₓMnₓSe nanoparticles were nearly monodispersed and uniform with a particle size 7.7 nm. FIG. 7B shows Manganese was contained in the particles. FIG. 7C shows a schematic of the surface covered with an organic layer of TOPO molecules.

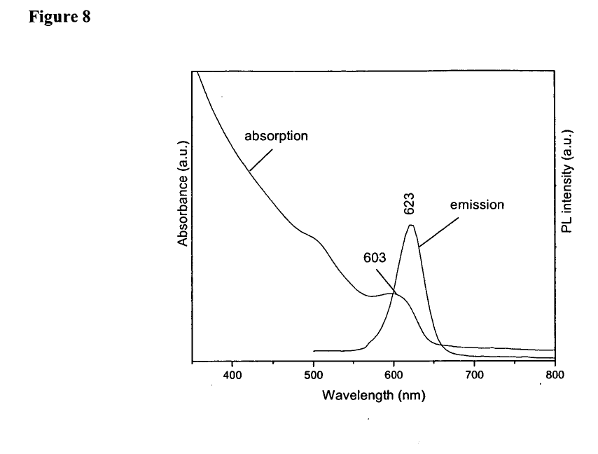

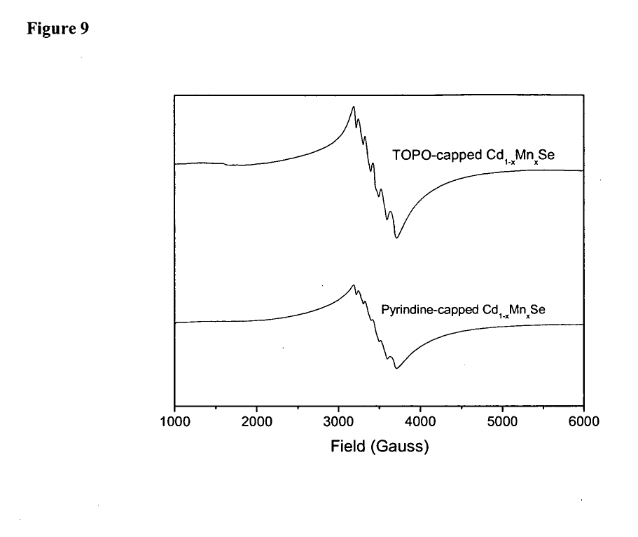

Optical and paramagnetic properties were also characterized and the results are shown in FIG. 8 and FIG. 9.

### EXAMPLE 2

### Doping Manganese in the Quantum Shell

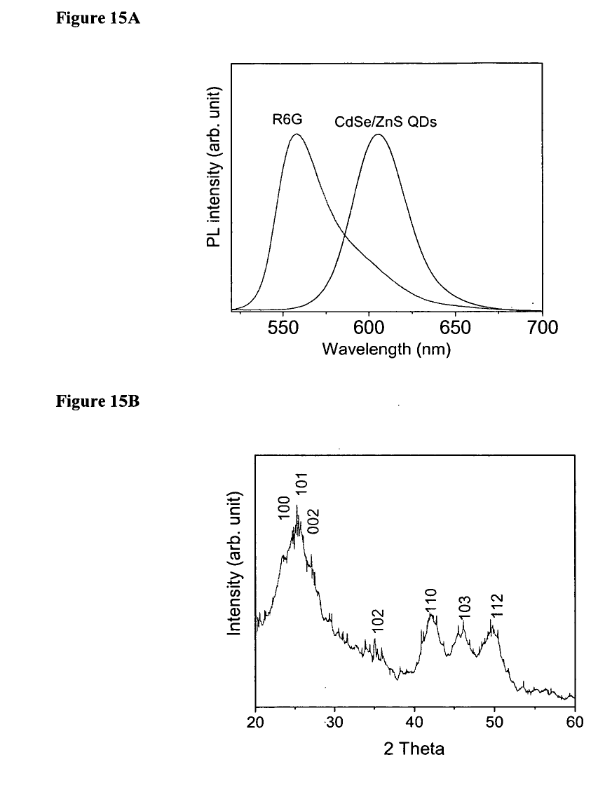

The above-mentioned method is an effective one for incorporating Manganese into CdSe nanoparticles. However, limitations exist in the low level of Mn-doping and the possibility of decreasing the photoluminescent efficiency. To overcome these problems, a new material containing a CdSe core and a manganese-doped ZnS (or CdS) shell was developed. Compared with Cd₁₋ₓMnₓSe particles, the new material has the following advantages: (1) Manganese could be doped with much higher concentration due to the comparable parameters of crystal lattice between Manganese and ZnS (or CdS), (2) there is improvement of the photoluminescent efficiency due to the ZnS (or CdS) shell, and (3) Manganese does not enter the crystal lattice of CdSe, accordingly, the doping will not affect the perfection of CdSe crystal lattice. FIG. 15A shows a comparison of the emission profiles between the dye of rhodamine 6 G and CdSe/ZnS nanoparticles. FIG. 15B depicts the XRD pattern which shows the wurtzite structure of CdSe nanoparticles.

A post-synthesis method has been used for the purpose. CdSe nanoparticles were synthesized at a high temperature using a known method in TOPO-HDA system (Mekis et al., 2003). A Mn-doped ZnS (or CdS) shell is subsequently grown by injecting the diethylzinc, dimethylmanganese, and H₂S gas (Zinc, Manganese, and Sulfur sources, respectively) at a temperature lower than 170° C.

Synthesis of CdSe/ZnMnS CdSe nanoparticles were synthesized at a high temperature using a known method in TOPO-HDA system (Mekis et al., 2003). 8 g of TOPO was dried and degassed under vacuum at 200° C. for 30 min in a 250 mL flask. Then, the system was cooled to 80-120° C. and flowing with Argon gas, 5 g of HDA was added, and the drying process was continued at 140° C. for 20 min. The stock solution of TOPSe prepared by dissolving 0.158 g of selenium in TOP was added and the mixture was heated to 300° C. The cadmium solution (0.12 g of cadmium acetate in 3 mL TOP) was quickly injected under vigorous stirring. Reaction temperature was then decreased to 260° C. for further growth of CdSe particles. After cooling to room temperature, CdSe particles were then isolated with solvent pair of chloroform and methanol and stocked with chloroform for further coating with Zn₁₋ₓMnₓS shell.

For the growth of the shell, a mixture of 14 g of TOPO and 9 g HDA (purified by the same procedures as that used in synthesis of CdSe particles) was heated to 170° C., and CdSe nanoparticles in chloroform solution were added, the solvent of chloroform was pumped off. The system was then put in argon atmosphere. A calculated amount of diethylzinc and dimethylmanganese was mixed in 3 mL of TOP, and then injected in drop into the reactor. H₂S gas was simultaneously injected into the mixture with a syringe in an interval of 10 min for 4 times (2 mL for each shot). The temperature was then decreased to and maintained at 150° C. for 1 hour.

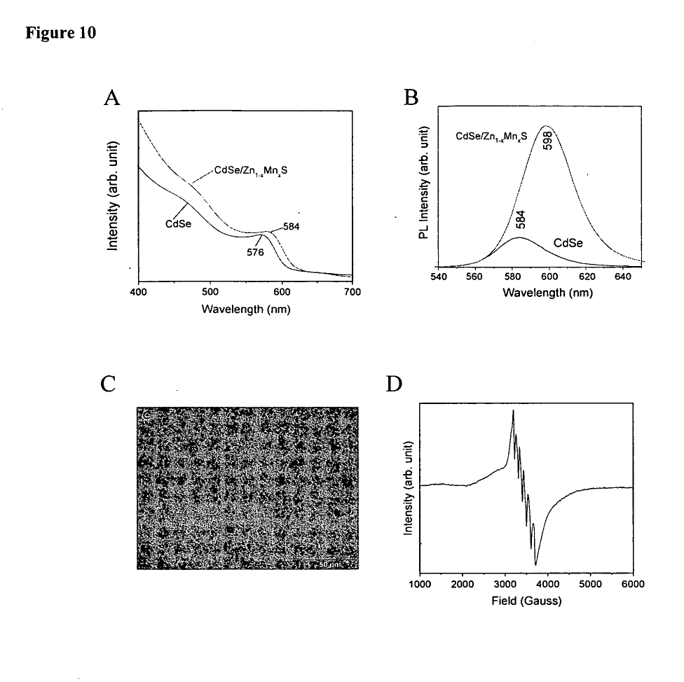

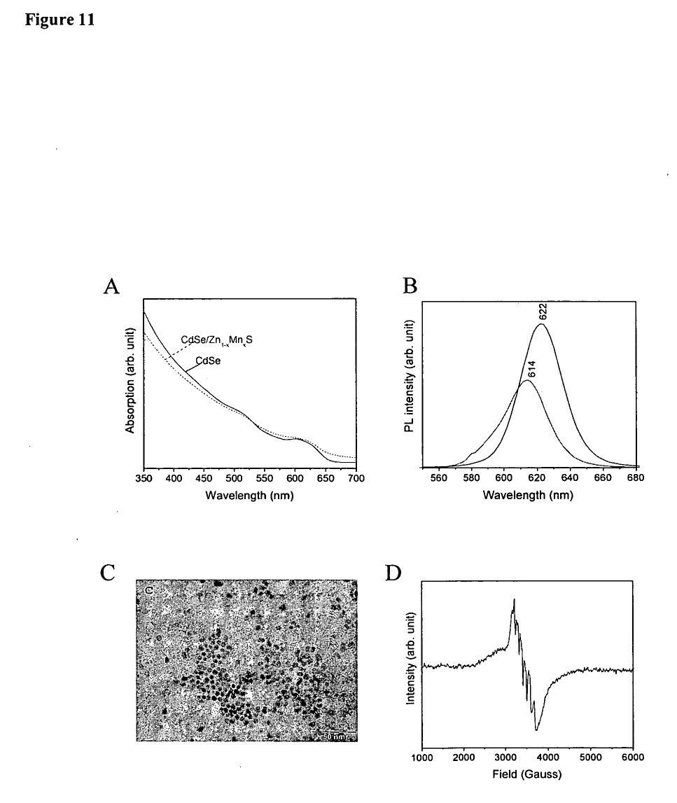

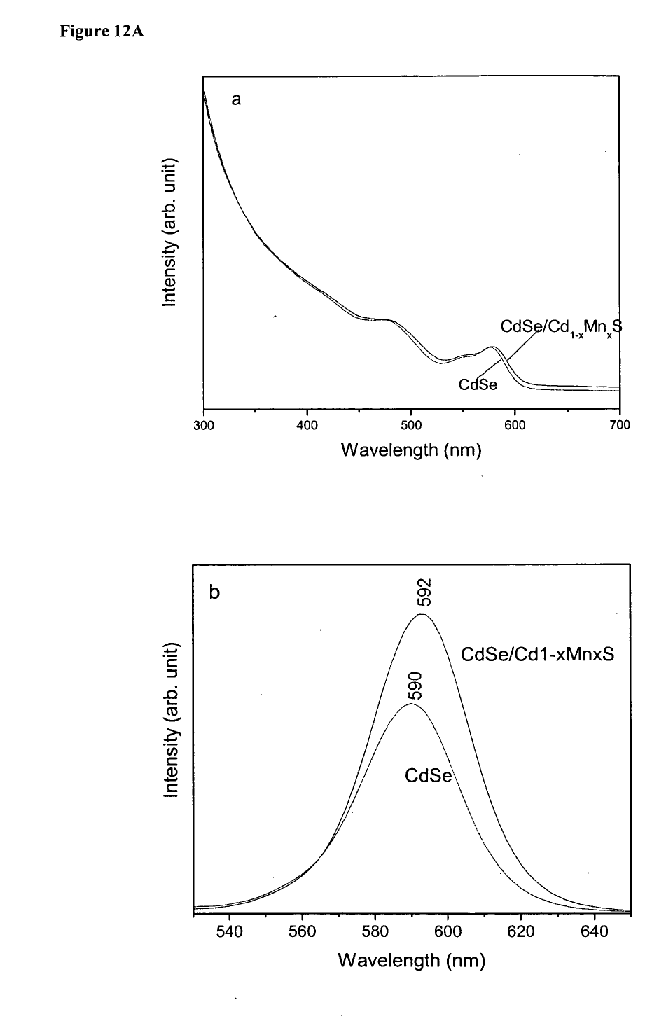

Results FIG. 10 shows some of the characterization results of ˜3 nm CdSe/Zn_{0.99}Mn_{0.01}S nanoparticles. In FIG. 10A, the band edge of core/shell nanoparticles shows red shift in comparison with that of naked CdSe nanoparticles. This red shift is indicative of the formation of a core/shell structure. As shown in FIG. 10B, PL spectra show red shift, and the intensity, which is normalized to the absorption spectrum, is considerably increased in the core/shell structure in comparison with that of the naked CdSe nanoparticles. EPR spectrum (FIG. 10D) shows a hyperfine six peaks, which indicate that the Manganese species was substantially incorporated into the ZnS lattice. The spectrum shows the pattern of six lines, which comes from the manganese hyperfine splitting (nuclear spin I=5/2). This feature of EPR pattern implies the contribution from Mn²⁺ substituted in Zn²⁺ site, isolated Manganese ions at the surface along with the Mn—Mn dipolar interaction inside the same particles. Also the broad signal might be due to the interacting Manganese centers (Borse et al., 1999). FIG. 11 shows some of the characterization results of ˜5 nm CdSe/Zn_{0.99}Mn_{0.01}S nanoparticles. The spectrum shows the pattern of six lines accompanied by smaller ones between them. The presence of small ones may be due to the higher concentration of Manganese species which contributes a stronger Mn—Mn interaction than that in the sample of CdSe/Zn_{0.99}Mn_{0.01}S. As shown in FIG. 11C, CdSe/Zn_{0.97}Mn_{0.03}S forms monodispersed and uniform nanoparticles.

Synthesis of CdSe/Cd₁₋ₓMnₓS nanoparticles CdSe nanoparticles were synthesized using the same procedures as that for synthesis of CdSe/Zn₁₋ₓMnₓS except for the molar ratio of Cadmium and Selenium controlled at 1:1. For the growth of the shell, a mixture of 14 g of TOPO and 9 g HDA (purified by the same procedures as that used in synthesis of Cadmium particles) was heated to 170° C., CdSe nanoparticles in chloroform solution were added, and the solvent of chloroform was pumped off. The system was then put in argon atmosphere. A calculated amount of dimethylmangnese was mixed in ˜1 mL of TOP, and then injected in drop into the reactor. H₂S gas was simultaneously injected into the mixture with a syringe in an interval of 10 min. The temperature was then decreased and maintained at 150° C. for 1 hour.

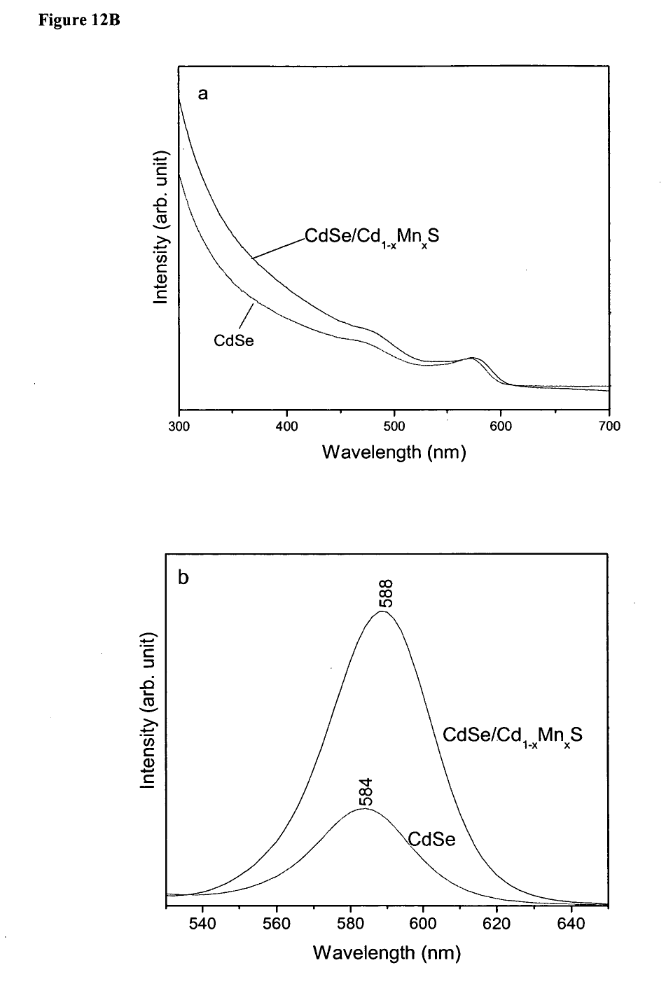

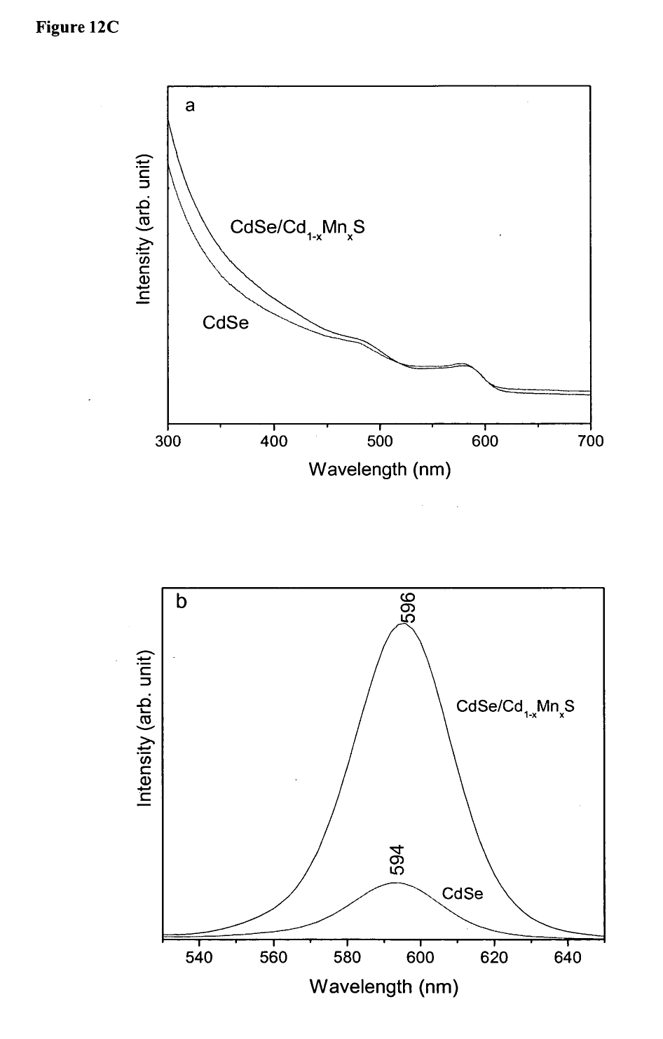

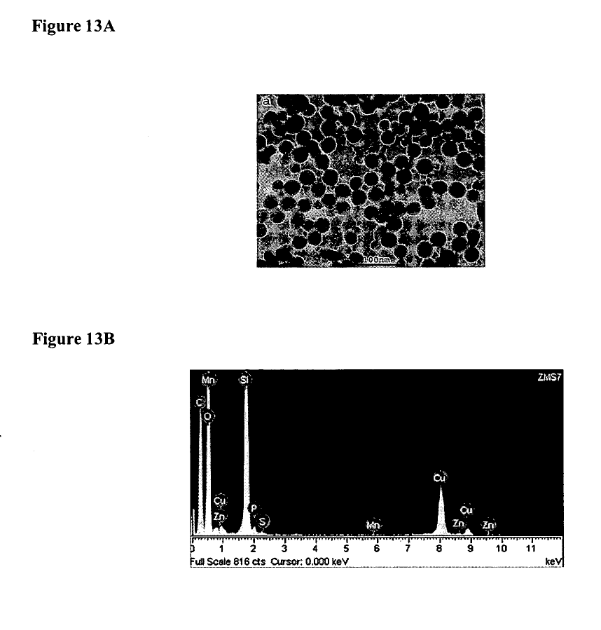

Results A few samples with different doped amount of manganese were also synthesized and characterized on their optical properties. As shown in FIG. 12, the PL efficiency increased after capping a Cd₁₋ₓMnₓS shell on CdSe core. The thickness of Cd₁₋ₓMnₓS shell might be from ˜1 to 3 monolayers, which can be further estimated from TEM results. PL efficiency increased with the thickness of the shell for the 3 samples. Absorption edge and the PL spectrum show a slightly red shift, and this may be explained by the following reasons: the absorption edge and the PL spectrum tend to shift towards red wavelength after the growth of a shell, however, the growth of the shell makes the CdSe core smaller (due to the substitution of Selenium by S), and the spectra tend towards blue-shift. The reverse tendency makes the spectra shift less than that in the CdSe/Zn₁₋ₓMnₓS material.

### EXAMPLE 3

### Preparation of Zn₁₋ₓMnₓS/SiO₂ Using Microemulsion Method

Strategy for the synthesis In typical synthesis, Zn₁₋ₓMnₓS nanoparticles form in a reverse microemulsion phase composed of nonionic surfactant olyoxyethylene nonaphenyl ether (Igepal CO-520). The synthesis involves the following procedures: a dilute (NH₄)₂S solution is added to one microemulsion solution while a dilute Zn(NO₃)₂ and Mn(NO₃)₂ solution is added to the other. After equilibration, the two aliquot microemulsions are mixed and allowed to stand overnight. The silica coating is generated by the addition of ammonium hydroxide and tetraethoxysilane to the Zn₁₋ₓMnₓS-contained microemulsion system. After 18 hours, the resulting Zn₁₋ₓMnₓS/SiO₂ nanoparticles are isolated by adding acetone to the microemulsion system followed by centrifugation and washing with cyclohexane and methanol for few times. Zn₁₋ₓMnₓS/SiO₂ nanoparticles of core-shell structure are thus obtained. To synthesize a nanocomposite of Zn₁₋ₓMnₓS homogeneously dispersed in silica a similar method was used. That is, simultaneously mixing three parts of microemulsion containing Zn²⁺, Mn²⁺ S²⁺, and ammonium hydroxide and tetraethoxysilane, respectively. In this way, the formation of Zn₁₋ₓMnₓS and silica proceed simultaneously, as a result, the semiconductor particles are homogeneously dispersed in silica nanoparticles.

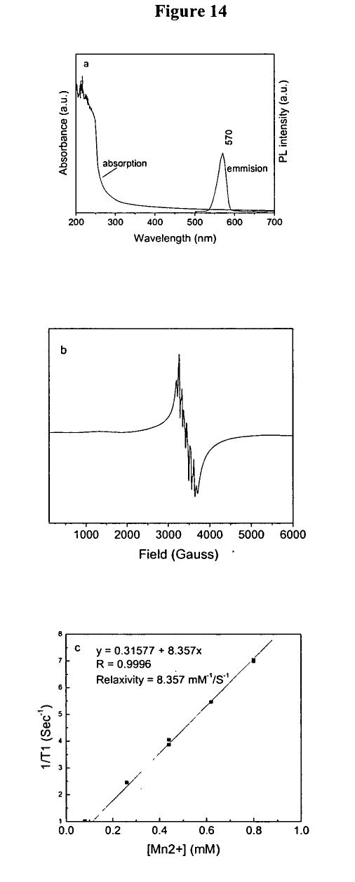

Results TEM micrograph (FIG. 1 3A) shows that the Zn₁₋ₓMnₓS/SiO₂ were uniform and monodispersed particles with an average particle size of around 50 nm. The sample showed a weak photoluminescent peak at 570 nm (FIG. 14A), characteristic peaks of manganese in EPR spectra (FIG. 14B), and T1 relaxation of 8.357 mM⁻¹/S⁻¹ (FIG. 14C).

### REFERENCES

1. Anderson, S. H. C.; Elliot, H.; Wallis, D. J.; Canham, L. T.; Powell, J. J. “Dissolution of different froms of partially porous Silicon wafers under simulated physiological conditions.” Phys. stat. sol. (a) 2003, 197, 331-335.
2. Baldwin, R. K.; Pettigrew, K. A.; Ratai, E.; Augustine, M. P.; Kauzlarich, S. M. “Solution reduction synthesis of surface stabilized Silicon nanoparticles” Chem. Comm. 2002, 1822-1823.
3. Bargik-Chory et al., (2003) Phys. Chem. Chem. Phys. 5:1639-1643.
4. Bol, A. A. et al. (2002) Chem Matter 14:1121-1126.
5. Borse, P. H.; Srinivas, D.; Shinde, R. F.; Date, S. K; Vogel, W.; Kulkarni, S. K.: Effect of Mn²⁺ concentration in ZnS nanoparticles on photoluminescence and electro-spin-resonance spectra. Physical Review B, 1999, 60, 8659-8664.
6. Bruchez, M. Jr, et al. Science, 1998, 281, 2013.
7. Chan, W. C. W. and Nie, S. (1998) Science 281:2016-2018.
8. Chan, W. C. W. et al. (2002) Curr. Op. Biotech 13:40-46.
9. Chan, W. C. W. et al. Science, 1998, 281, 2016. [1] Cao, L.; Zhang, J.; Ren, S.; Huang, S.: Luminescence enhancement of core-shell ZnS:Mn/ZnS nanoparticles. Appl. Phys. Lett. 2002, 23, 4300-4302.
10. Dabbousi BO et al. (1997) J. Phys. Chem. 101:9463-9475.
11. Dinsmore, A. D. et al. (2000) J. Applied Phys. 88(9):4985-4993.
12. Dubertret, B.; Skourides, P.; Norris, D. J.; Noireaux, V.; Brivanlou, A. H.; Libchaber, A.: In vivo imaging of quantum dots encapsulated in phospholipid micelles. Science 2002, 298, 1759-1762.
13. Ethiraj, A. S. et al. (2003) J. Chem. Phys. 118(19):8945-8953.
14. Gao, X. et al. (2002) J. Biomed. Optics 7(4):532-537.
15. Gao, X. H.; Cui, Y.; Levenson, R. M.; Chung, W. K.; Nie, S. M.: In vivo cancer targeting and imaging with semiconductor quantum dots. Nature biotechnol. 2004, 22, 969-976.
16. Gerion, D.; Pinaud, F.; Williams, S. C.; Parak, W. J.; Zanchet, D.; Weiss, S.; Alivisatos, A. P.: Synthesis and Properties of Biocompatible Water-Soluble Silica-Coated CdSe/ZnS Semiconductor Quantum Dots. J. Phys. Chem. B 2001 105, 8861-8871.
17. Hines, M. A. et al. (1996) J. Phys. Chem. 100:468-471.
18. Jaiswal, J. K. et al. (2003) Nature Biotech. 21:47-51.
19. Jang, H.; Pell, L. E.; Korgel, B. A.; English, D. S. “Photoluminescence quenching of Silicon nanoparticles in phospholipid vesicle bilayers” Journal of Photochemistry and Photobiology A: Chemistry 2003, 158, 111-117.
20. Ji, J.; Chen, Y.; Senter, R. A.; Coffer, J. L. “Surface Modification of Erbium-doped Silicon Nanocrystals” Chem. Mater. 2001, 13, 4783-4786.
21. Khosravi, A. A.; Kundu, M.; Kuruvilla, B. A.; Shekhawat, G. S.; Gupta, R. P.; Sharma, A. K.; Vyas, P. D.; Kulkarni, S. K.: Manganese doped zinc sulphide nanoparticles by aqueous method. Appl. Phys. Lett. 1995, 67, 2506-2508.
22. Kirchner, C.; Liedl, T.; Kudera, S.; Pellegrino, T.; Munoz Javier, A.; Gaub, H. E.; Stolzle, S.; Fertig, N.; Parak, W. J.; Cytotoxicity of Colloidal CdSe and CdSe/ZnS Nanoparticles. Nano Lett.; (Communication); 2005; 5(2); 331-338
23. Kulkarni SK (2001) Appl. Surf. Sci. 169-170:438-446.
24. Larson, et al. (2003) Science 300:1434.
25. Leeb, J.; Gebhardt, V.; Muller, G.; Haarer, D.; Su, D.; Giersig, M.; McMahon, G.; Spanhel, L.: Colloidal synthesis and electroluminescence properties of nanoporous (MnZnS)—Zn-II films. J. Phys. Chem. B 1999, 103, 7839-7845.
26. Levy (1996) J. Phys. Chem. 100:18322-18326.
27. Levy, L. et al. (1996) Langmuir 15:3386-3389.
28. Li, Z. F.; Swihart, M. T.; Ruckenstein, E. “Luminescent Silicon Nanoparticles Capped by Conductive Polyaniline through the Self-Assembly Method” Langmuir 2004, ASAP.
29. Z. F. Li, M. T. Swihart, and E. Ruckenstein, “Luminescent Silicon Nanoparticles Capped by Conductive Polyaniline through the Self-Assembly Method”, LANGMUIR 20 (5): 1963-1971 MAR 2 2004
30. Liu, Q.; Kauzlarich, S. “A new synthetic route for the synthesis of hydrogen terminated Silicon nanoaparticles” Mat Sci Eng B-Solid State Matls for Adv Tech 2002, 96, 72-75.
31. Mekis, I.; Talapin, D. V.; Komowshi, A.; Haase, M.; Weller, H.: One-Pot synthesis of highly luminescent CdSe/CdS core-shell nanocrytals via orgranometllic and “greener” chemical approaches. J. Phys. Chem. B, 2003, 107, 7454-7462.
32. Mikulec, F. V.; Kuno, M.; Bennati, M.; Hall, D. A.; Griffin, R. G.; Bawendi, M. G.: Organometallic Synthesis and Spectroscopic Characterization of Manganese-Doped CdSe Nanocrystals. J. Am. Chem. Soc, 2000, 122, 2532-2540.
33. Padadimitrakopoulos, F.; Phely-Bobin, T.; Wisniecki, P. “Self-assembled nanoSilicon/siloxane composite film” Chem. Mater. 1999, 11, 522-525.
34. Parak, W. J.; Gerion, D.; Pellegrino, T.; AZanchet, D.; Micheel, C.; Williams, S. C.; Boudreau, R.; Le Gros, M. A.; Larabell, C. A.; Alivisatos, A. P. “Biological applications of colloidal nanocrystals” Nanotechnology 2003, 14, R15-R27.
35. Pathak, S. et al. (2001) 123:4103-4104.
36. Peng, X et al. (1997) JACS 119:7019-7029.
37. Sooklal, K.; Cullum, B. S.; Angel, S. M.; Murphy, C J.: Photophysical properties of ZnS nanoclusters with spatially localized Mn2+. J. Phys. Chem., 1996, 100, 4551-4555.
38. Sukhanova, A.; Venteo, L.; Devy, J.; Artemyev, M.; Oleinikov, V.; Pluot, M.; Nabiev, I.: Highly stable fluorescent nanocrystals as a novel class of labels for immunohistochemical analysis of paraffin-embedded tissue sections. Lab Invest. 2002, 82, 1259-61.
39. Sutherland, A. J. Current Opinion in Solid State and Materials. Science, 2002, 6, 365.
40. Wang, L.; Reipa, V.; Blasic, J. “Silicon Nanoparticles as a Luminescent Label to DNA” Bioconjugate Chemistry 2004, 15, 409-412.
41. Wang, Y. A.; Li, J. J.; Chen, H.; Peng, X.: Stabilization of inorganic nanocrystals by organic dendrons. J. Am. Chem. Soc.; 2002; 124. 2293-2298.
42. Warburton, R. J.; et al. Phys. Rev. Lett. 1997, 79, 5282.
43. Warren C W, Nie S “Quantum Dot Bioconjugates for Ultrasensitive Nonisotopic Detection”. Science. 1998. 281:2016-2018.
44. Wolfgang, J. P.; et al. Nanotechnology, 2003, 14, R15.
45. Zou, J.; Baldwin, R.; Pettigrew, R. “Solution synthesis of ultrastable luminescent siloxane-coated Silicon nanoparticles” Nano Letters 2004, 4, 1181-1186.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

1. An imaging agent which is detectable by both optical imaging and magnetic resonance imaging (MRI).

2. The imaging agent of claim 1 wherein the imaging agent is a quantum dot.

3. The imaging agent of claim 1 wherein the imaging agent comprises an optically active core and a shell comprising a magnetic material.

4. The imaging agent of claim 3 wherein the core comprises a luminescent nanoparticle.

5. The imaging agent of claim 4 wherein the luminescent nanoparticle is a Group II-VI nanoparticle, a Group III-V nanoparticle or a Silicon nanoparticle.

6. The imaging agent of claim 5 wherein the Group II element is selected from the group consisting of Zinc, Cadmium, and Manganese and the Group VI element is selected from the group consisting of Sulfur, Selenium, and Tellurium.

7. The imaging agent of claim 3 wherein the magnetic material is selected from the group consisting of Manganese (II), Iron (III), Lanthanum, Gadolinium, and other magnetic Lanthanides.

8. The imaging agent of claim 3 wherein the core is a Silicon core and the shell is a SiOₓ shell.

9. The imaging agent of claim 8 wherein the SiOₓ, shell is produced by silanization of the surface of a Silicon nanoparticle core.

10. The imaging agent of claim 3 wherein the shell further comprises a matrix, wherein the matrix components are selected from the group consisting of Zinc Sulfide, Cadmium Sulfide and Silicon.

11. The imaging agent of claim 10 wherein the core comprises Cadmium Selenide, the matrix comprises Zinc Sulfide, and the magnetic material is Manganese (II).

12. The imaging agent of claim 2 wherein the imaging agent further comprises a water soluble coating.

13. The imaging agent of claim 12 wherein the water soluble coating is formed by treatment of the imaging agent with a treatment or agent selected from the group consisting of mercaptoalkanoic acid ligands, PEG, silanization, phospholipid micelles, organic dendrons, cysteines, and amphipathic polymers.

14. The imaging agent of claim 3 wherein the imaging agent further comprises a moiety attached to the shell comprising a functional group selected from the group consisting of amine, carboxy, and thiol.

15. A method for imaging a biological sample with an imaging agent comprising:
contacting the biological sample with the imaging agent, and
detecting the imaging agent by optical detection or MRI detection.

16. The method of claim 15 wherein said imaging agent is a quantum dot.

17. The method of claim 15 wherein the detection is by optical detection and by MRI detection.

18. The method of claim 15 wherein the imaging agent comprises an optically active core and a shell comprising a magnetic material, wherein the core comprises a luminescent nanoparticle, wherein the luminescent nanoparticle is a Group II-VI nanoparticle, a Group III-V nanoparticle or a Silicon nanoparticle, wherein the Group II element is selected from the group consisting of Zinc, Cadmium, and Manganese and the Group VI element selected from the group consisting of Sulfur, Selenium, and Tellurium, wherein the magnetic material is selected from the group consisting of Manganese (II), Iron (III), Lanthanum, Gadolinium, and other magnetic Lanthanides, wherein the shell further comprises a matrix, wherein the matrix components are selected from the group consisting of Zinc Sulfide, Cadmium Sulfide and Silicon.

19. The method of claim 18 wherein the imaging agent further comprises a water soluble coating, wherein the water soluble coating is formed by treatment of the imaging agent with a treatment or agent selected from the group consisting of mercaptoalkanoic acid ligands, PEG, silanization, phospholipid micelles, organic dendrons, cysteines, and amphipathic polymers.
